(19) United States
(12) Patent Application Publication
(10) Pub. No.: US 2009/0326414 A1
(43) Pub. Date: Dec. 31, 2009

(54) CYTOLOGICAL SAMPLING BRUSH

(75) Inventor: Eric Peltier, Clamart (FR)

Correspondence Address: YOUNG & THOMPSON, 209 Madison Street, Suite 500, Alexandria, VA, 22314, US

(73) Assignee: NOVACYT, VELIZY VILLACOUBLAY (FR)

(21) Appl. No.: 12/374,841

(22) PCT Filed: Jul. 4, 2007
(86) PCT No.: PCT/FR07/01135

§ 371 Date: Jan. 23, 2009

(30) Foreign Application Priority Data
Jul. 26, 2006 (FR) 0606856

(51) Int. Cl. A61B 10/02

(52) U.S. Cl. 600/569

(57) ABSTRACT

A cytological sampling brush includes a manipulation handle (2) at one of end of which are provided a first portion in the form of exocol sampling brush (4) and a second portion in the form of endocol sampling swab (5) at the center of the first portion in brush (4) form. This brush is characterized in that the second portion in swab (5) form is attached at the end of the handle (2) and is removable from the first portion by sliding in a hole (6) of the first portion in brush (4) form, provided with elements (7) for retaining cells for collecting cells from the second portion in swab (5) form, during retraction of the first portion in brush (4) form.

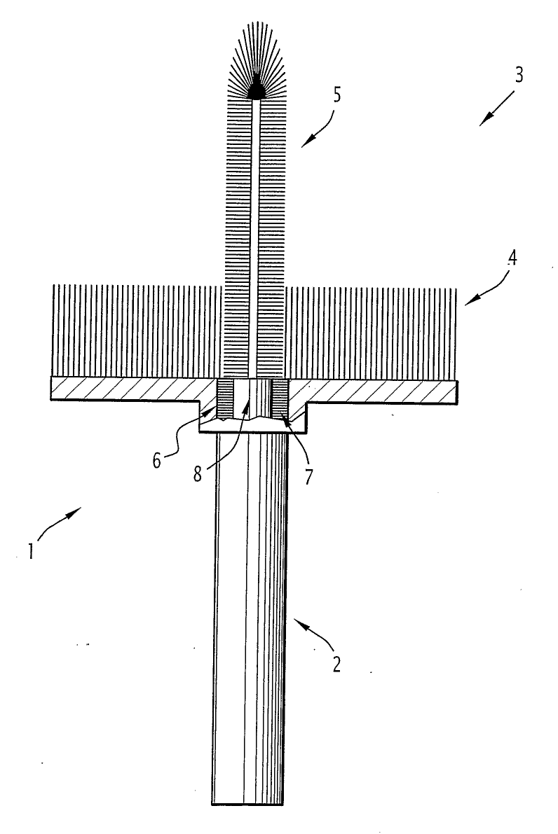

The present invention relates to a cytological sampling brush.

More specifically, the invention relates to such a brush which comprises an operating handle, at one of the ends of which there are provided a first portion in the form of an exocervical sampling brush and a second portion in the form of an endocervical sampling scraper at the centre of the first brush-like portion.

Sampling brushes currently known in the prior art have a given number of disadvantages since the scraper-like portion is not generally of sufficient length and is excessively flexible to ensure correct sampling of cells at the endocervix or the exocervix/endocervix junction region.

Incorrect sampling of cells may lead to subsequent analysis of those cells being incomplete with the result that this analysis will not reveal any problem.

Therefore, an object of the invention is to overcome those problems.

To that end, the invention relates to a cytological sampling brush of the type comprising an operating handle, at one of the ends of which there are provided a first portion in the form of an exocervical sampling brush and a second portion in the form of an endocervical sampling scraper at the centre of the first brush-like portion, characterised in that the second scraper-like portion is fixed to the end of the handle and is detachable from the first portion by sliding into a hole of the first brush-like portion, which hole is provided with means for retaining cells allowing the collection of the cells from the second scraper-like portion when it is withdrawn from the first brush-like portion.

According to other features of the invention:
the first brush-like portion is fixed to the handle by detachable fixing means;
the detachable fixing means comprise bayonet-type fixing means; and
the retention means of the hole of the first brush-like portion comprise comb-like means, through which the second scraper-like portion passes when it is withdrawn from the first brush-like portion.

The invention will be better understood from a reading of the following description which is given purely by way of example and with reference to the appended drawing which is a schematic, partially sectioned view of a cytological sampling brush according to the invention.

This FIGURE illustrates a cytological sampling brush which is generally designated 1 and which comprises an operating handle generally designated 2, at one of the ends of which there are provided brush-like means generally designated 3.

These means comprise a first portion in the form of an exocervical sampling brush which is generally designated 4 and a second portion in the form of an endocervical sampling scraper which is generally designated 5 and which is provided, for example, at the centre of the first brush-like portion 4.

According to the invention, the second scraper-like portion generally designated 5 is fixed to the end of the handle 2 and is detachable from the first portion by sliding into a hole which is generally designated 6 of this first brush-like portion. The hole 6 is provided with means 7 for retaining cells allowing collection of the cells from the second scraper-like portion when it is withdrawn by sliding into the hole of the first brush-like portion.

The retention means 7 may be constituted by comb-like means which allow the cells to be retained when the second scraper-like portion is withdrawn.

It should also be noted that the first brush-like portion can be fixed to the handle 2 by means of detachable fixing means which are generally designated 8 in this FIGURE and which comprise, for example, bayonet-type fixing means.

Different conventional means which are known in the prior art may be envisaged in order to ensure that those various components are retained in position relative to each other during use, such as, for example, braking means, means for passing an over-centre position, etcetera.

It will be appreciated that this sampling brush has a given number of advantages over those of the prior art, in particular by allowing the construction of the two brush portions, that is to say, the portion in the form of a brush and the portion in the form of a scraper, in a distinct and separate manner in order to confer on them in particular the characteristics relating to length and flexibility required by each of them specifically.

This therefore allows an improvement in the sampling carried out and the quality of the corresponding analyses.

Naturally, other embodiments may be envisaged.

1. Cytological sampling brush, of the type comprising an operating handle (2), at one of the ends of which there are provided a first portion in the form of an exocervical sampling brush (4) and a second portion in the form of an endocervical sampling scraper (5) at the centre of the first brush-like portion (4), characterised in that the second scraper-like portion (5) is fixed to the end of the handle (2) and is detachable from the first portion by sliding into a hole (6) of the first brush-like portion (4), which hole is provided with means (7) for retaining cells allowing the collection of the cells from the second scraper-like portion (5) when it is withdrawn from the first brush-like portion (4).

2. Brush according to claim 1, characterised in that the first brush-like portion (4) is fixed to the handle (2) by detachable fixing means (8).

3. Brush according to claim 2, characterised in that the detachable fixing means comprise bayonet-type fixing means (8).

4. Brush according to claim 1, characterised in that the retention means (7) of the hole of the first brush-like portion (4) comprise comb-like means, through which the second scraper-like portion (5) passes when it is withdrawn from the first brush-like portion (4).

5. Brush according to claim 2, characterised in that the retention means (7) of the hole of the first brush-like portion (4) comprise comb-like means, through which the second scraper-like portion (5) passes when it is withdrawn from the first brush-like portion (4).

6. Brush according to claim 3, characterised in that the retention means (7) of the hole of the first brush-like portion (4) comprise comb-like means, through which the second scraper-like portion (5) passes when it is withdrawn from the first brush-like portion (4).
